(19) United States
(12) Patent Application Publication
(10) Pub. No.: US 2019/0216321 A1
(43) Pub. Date: Jul. 18, 2019

(54) Detection and Response System for Opioid Overdoses

(71) Applicant: Vincenzo Grande, Bridgewater, NJ (US)

(72) Inventor: Vincenzo Grande, Bridgewater, NJ (US)

(21) Appl. No.: 15/873,445

(22) Filed: Jan. 17, 2018

(51) Int. Cl. A61B 5/00; A61M 5/142; A61B 5/024; A61M 5/32; G01N 33/94

(52) U.S. Cl. CPC A61B 5/0024; A61B 5/6823; A61B 5/681; G01N 33/9486; A61B 5/024; A61B 5/486; A61M 5/326; A61M 5/14248

(57) ABSTRACT

A wearable system detects an opioid overdose and transmits a distress message with the wearer's GPS coordinates to one or more emergency response contacts. Concurrently, the system wirelessly signals a relay which energizes a solenoid injector, causing a prescribed dosage of an opioid antidote to be injected by a syringe into the wearer's body through a subcutaneous cannula, preferably located on the wearer's wrist. Detection of an opioid overdose is based on a symptomatic depressed heart rate, which is measured by a monitor worn on the chest.

### FIELD OF INVENTION

The present invention relates to the fields of systems for remotely monitoring the vital signs of a subject and for initiating emergency response measures when monitored vital signs indicate a life-threatening situation.

### BACKGROUND OF THE INVENTION

In recent years, deaths from overdoses of opioid drugs have reached epidemic proportions, claiming tens of thousands of lives each year. Wearable GPS devices are available to track the whereabouts of drug abusers and to monitor vital signs, such as depressed respiratory and heart rates, which are indicative of an opioid overdose. Once an overdose situation is detected, however, the time for emergency responders to reach the subject and administer an opioid antidote is often too long to save his/her life. Therefore, there is a need for a system which combines remote detection of an opioid overdose with remote activation of a wearable antidote injection system upon detection of an overdose situation.

### SUMMARY OF THE INVENTION

The present invention is a wearable system for detecting an opioid overdose and, upon such detection, transmitting a distress message with the wearer's GPS coordinates to one or more emergency responders. Concurrently with transmitting the message to emergency responders, the system wirelessly signals a relay which energizes a solenoid injector, causing the injector to depress the plunger of a syringe containing a prescribed dosage of an opioid antidote, such as naloxone hydrochloride. The needle of the syringe is set in a subcutaneous cannula, such that, upon depression of the syringe plunger by the solenoid injector, the syringe injects the wearer with the prescribed dosage of the opioid antidote.

The detection of an opioid overdose in the wearer is based upon a depressed heart rate, which is symptomatic of opioid use. Preferably, the system comprises a monitor unit, which is removably attached to the wearer's chest by a band, strap or adhesive strips, and an injector unit, which can be removably attached to one of the wearer's arms or legs, preferably to one of the wearer's wrists, also by a band, strap or adhesive strips.

The foregoing summarizes the general design features of the present invention. In the following sections, specific embodiments of the present invention will be described in some detail. These specific embodiments are intended to demonstrate the feasibility of implementing the present invention in accordance with the general design features discussed above. Therefore, the detailed descriptions of these embodiments are offered for illustrative and exemplary purposes only, and they are not intended to limit the scope either of the foregoing summary description or of the claims which follow.

### BRIEF DESCRIPTION OF THE DRAWINGS

FIG. 1 is a perspective view of one embodiment of the present invention attached to the body of a wearer;

FIG. 2 is a schematic diagram of the components of one embodiment of the present invention and their interaction;

FIG. 3 is a cross-sectional view of a de-energized solenoid injector, a syringe and a subcutaneous cannula, according to one embodiment of the present invention;

FIG. 4 is a cross-sectional view of an energized solenoid injector, a syringe and a subcutaneous cannula, according to one embodiment of the present invention; and

FIG. 5 is a flow chart depicting the operation of one embodiment of the present invention.

### DETAILED DESCRIPTION OF THE PREFERRED EMBODIMENT

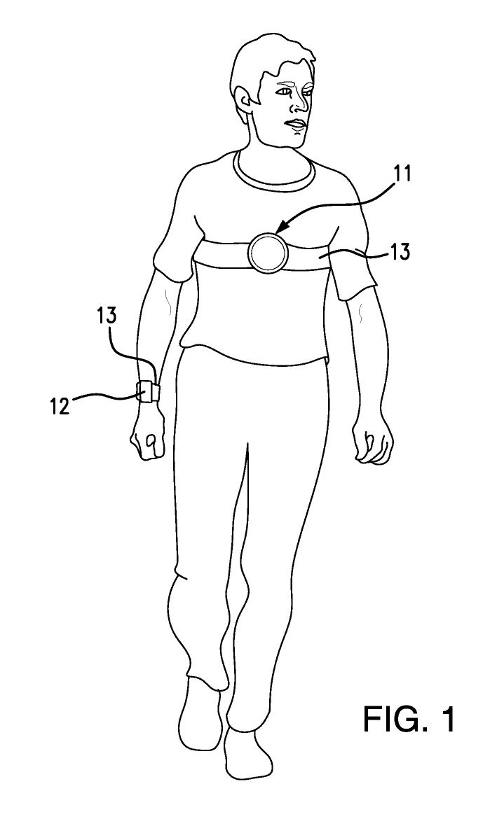

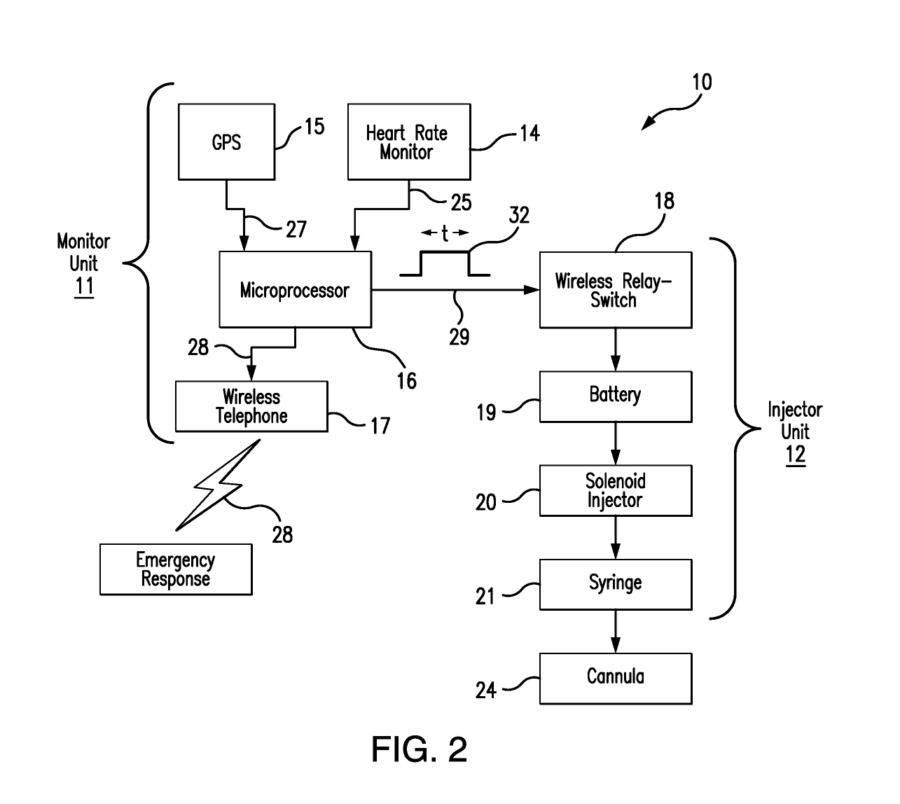

Referring to FIGS. 1 and 2, the preferred embodiment of the present invention 10 consists of a wearable monitor unit 11 and a wearable injector unit 12. Preferably, the monitor and injector units 11 12 are separate units removably attached to different parts of the wearer's body. Optimally, the monitoring unit 11 is worn across the chest, as shown in FIG. 1, supported by a strap, band 13 or adhesive strips, while the injector unit 12 is optimally worn around one of the wearer's wrists, as depicted in FIG. 1, also supported by a strap, band 13 or adhesive strips.

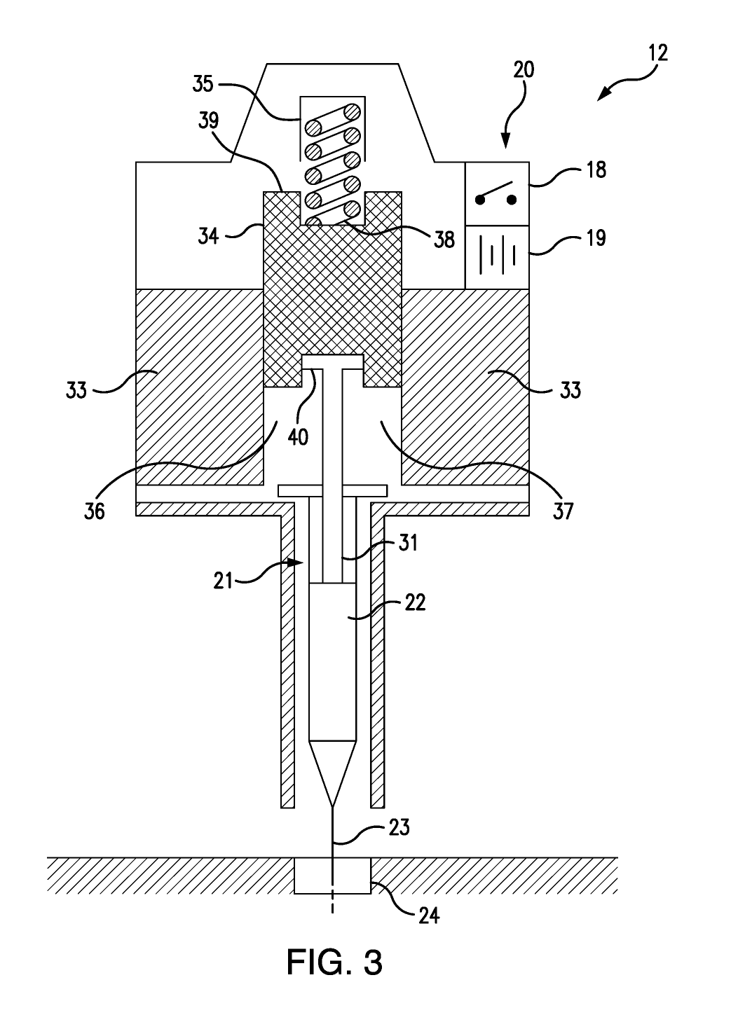

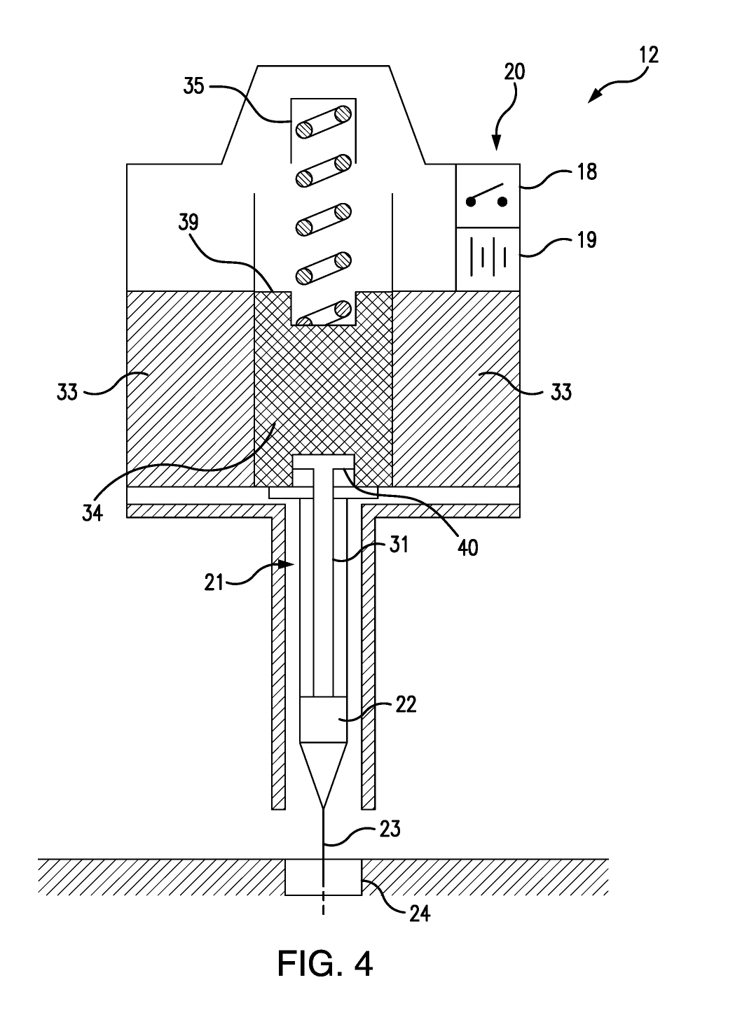

The monitor unit 11 comprises a heart rate monitor 14, a GPS 15, a microprocessor 16, and a wireless telephone transmitter 17. The injector unit comprises a wireless relay switch 18, a battery 19, a solenoid injector 20, and a syringe 21 containing a prescribed dosage of an opioid antidote 22, such as naloxone hydrochloride. As best seen in FIGS. 3 and 4, the syringe needle 23 is set in a subcutaneous cannula 24, through which the antidote 22 is injected into the wearer's body.

The heart rate monitor 14 continuously monitors the wearer's heart rate and continuously transmits 25 the wearer's heart rate, preferably in a digital format, to the microprocessor 26. The microprocessor 26 is programmed to continuously compare the wearer heart rate with a pre-set threshold heart rate indicative of an opioid overdose. Upon determining that the wearer's heart rate is below the threshold heart rate, the microprocessor is programmed to obtain the wearer's current location coordinates 27 from the GPS 15, and to send one or more emergency distress messages 28 to one or more emergency contacts, using the wireless telephone transmitter 17. The emergency messages 28 can be voice, text or a combination of both.

Upon determining that the wearer's heart rate is below the threshold heart rate, the microprocessor is also programmed to wirelessly transmit an activation signal 29 to the wireless relay switch 18 of the injector unit 12. Preferably bluetooth or rf, the activation signal 29 has a signal duration t 32, after which the signal 29 terminates. Upon receiving the activation signal 29, the relay switch 18 is configured to complete an energizing circuit by which the battery 19 energizes the solenoid injector 21 for the signal duration 32.

When energized, the solenoid injector 20 is configured to depress the syringe plunger 31, such that the syringe 21 injects the prescribed antidote dosage 22 subcutaneously through the cannula 24, as best seen in FIG. 4. At the end of the signal duration 32, the relay switch 18 is configured to open the energizing circuit, so that the battery 19 ceases to energize the solenoid injector 20. Upon being de-energized, the solenoid injector 20 is configured to lift the syringe plunger 31, so that the syringe 21 stops injecting the opioid antidote 22 into the wearer.

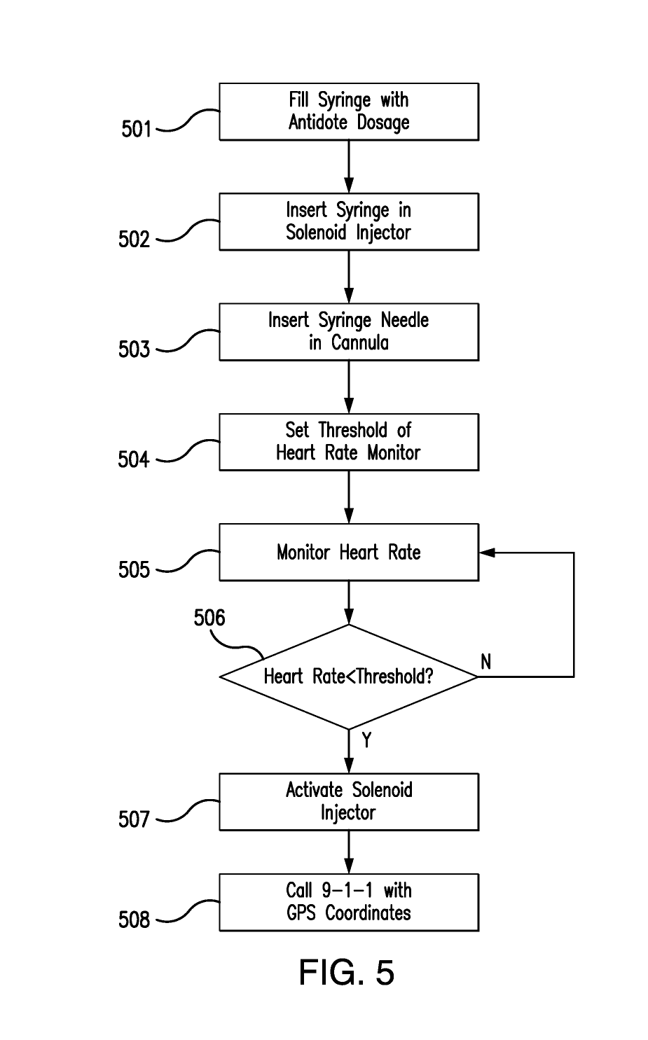

Referring to FIGS. 4 and 5, the solenoid injector 20 comprises an annular, cylindrical solenoid coil 33, a cylindrical, steel armature 34, and a solenoid spring 35. The solenoid coil 33 has an open central core 36, and the armature 34 is configured to slide within a cylindrical armature guide passage 37, which is axially aligned with the central core 36. The solenoid spring 35 has a proximal end 38 which is attached to the distal end 39 of the armature 34, so that the solenoid spring 35 urges the armature 34 away from the central core 36 of the solenoid coil 33.

When the solenoid coil 33 is energized, the armature 34 is magnetically drawn into the central core 36, as shown in FIG. 4, thereby depressing the syringe plunger 31, which is removably attached to the proximal end 40 of the armature 34, so as to cause the antidote 22 to be injected through the syringe needle 23 into the subcutaneous cannula 24 and into the wearer's body. When the solenoid coil 33 is de-energized, as shown in FIG. 3, the armature 34 is drawn out of the central core 36 by the retraction of the solenoid spring 35, which is no longer opposed by the magnetic field of the solenoid coil 33. The retracting armature 34 thereby lifts the syringe plunger 31 and thus terminates the antidote injection.

FIG. 5 depicts the process by which the system 10 is employed. The syringe is filled with the antidote dosage 501, and the loaded syringe is inserted into the solenoid injector 502. The syringe needle is next inserted into the cannula 503, and the threshold heart rate is set in the heart rate monitor 504. The heart rate is then monitored 505, and when determined to be below the threshold 506, triggers the activation of the solenoid injector 507 and an emergency call with the wearer's GPS coordinates 508.

Although the preferred embodiment of the present invention has been disclosed for illustrative purposes, those skilled in the art will appreciate that many additions, modifications and substitutions are possible, without departing from the scope and spirit of the present invention as defined by the accompanying claims.

1. A wearable system for detecting an opioid overdose and automatically administering to a wearer an opioid antidote, the system comprising:
a wearable monitor unit, comprising a heart rate monitor, a GPS, a microprocessor, and a wireless telephone transmitter;
a wearable injector unit, comprising a wireless relay switch, a battery, a solenoid injector, and a syringe containing a prescribed dosage of the opioid antidote;
wherein the monitor unit is configured to be removably attached to the wearer, such that the heart rate monitor continuously monitors a wearer heart rate and continuously transmits the wearer heart rate to the microprocessor;
wherein the microprocessor is programmed to continuously compare the wearer heart rate with a pre-set threshold heart rate indicative of an opioid overdose;
wherein the microprocessor is programmed, upon determining that the wearer heart rate is below the threshold heart rate, to obtain current wearer location coordinates from the GPS and to send one or more emergency messages, including the current wearer location coordinates, to one or more emergency contacts using the wireless telephone transmitter;
wherein the microprocessor is programmed, upon determining that the wearer heart rate is below the threshold heart rate, to wirelessly transmit an activation signal, having a signal duration, to the wireless relay switch of the injector unit;
wherein, upon receiving the activation signal, the wireless relay switch is configured to complete an energizing circuit, such that the battery energizes the solenoid injector during the signal duration;
wherein the solenoid injector is configured, upon being energized, to depress a plunger of the syringe, such that the syringe injects the prescribed dosage of the opioid antidote into the wearer;
wherein the wireless relay switch is configured, at the end of the signal duration, to open the energizing circuit, such that the battery ceases to energize the solenoid injector and the solenoid injector is de-energized; and
wherein the solenoid injector is configured, upon being de-energized, to lift the plunger of the syringe, such that the syringe ceases to inject the opioid antidote into the wearer.

2. The system according to claim 1, wherein the solenoid injector comprises an annular, cylindrical solenoid coil having an open central core, a cylindrical steel armature, which is configured to slide within a cylindrical armature guide passage that is axially aligned with central core of the solenoid coil, and a solenoid spring having a proximal end which is attached to a distal end of the armature, such that the solenoid spring biases the armature away from the central core of the solenoid coil, and wherein a proximal end of the armature is removably attached to the plunger of the syringe, and wherein, when the solenoid coil is energized, the armature is magnetically drawn into the central core of the solenoid coil, thereby depressing the plunger of the syringe, and wherein, when the solenoid coil is de-energized, the armature is drawn out of the central core of the solenoid coil by a retraction of the solenoid spring, thereby lifting the plunger of the syringe.

3. The system according to claim 1, wherein the syringe injects the specified dosage of the opioid antidote into the wearer through a cannula.

4. The system according to claim 2, wherein the syringe injects the specified dosage of the opioid antidote into the wearer through a cannula.
